(19) United States
(12) Patent Application Publication
(10) Pub. No.: US 2002/0069890 A1
(43) Pub. Date: Jun. 13, 2002

(54) Method for changing color of hair locks and apparatus for performing said method

(76) Inventors: Rainer Schweickert, Griesheim (DE); Peter Scheunert, Dieburg (DE)

Correspondence Address: STRIKER, STRIKER & STENBY, 103 East Neck Road, Huntington, NY, 11743, US

(21) Appl. No.: 09/996,494

(22) Filed: Nov. 29, 2001

(30) Foreign Application Priority Data
Dec. 13, 2000 (DE) 100 62 132.5

(51) Int. Cl. A61K 7/135; A45D 19/18

(52) U.S. Cl. 132/208; 132/270

(57) ABSTRACT

A method for changing hair lock color includes first placing a protective foil (7) on a flat hair lock support (4) and a hair lock (2) on the protective foil (7). Then a treatment composition (3) is applied to the hair lock (2) on the protective foil (7). After that a free portion of the protective foil (7) is folded and laid on the hair lock (2). Then a hot plate (6) is placed on and heats the hair lock (2). After that the treatment composition (3) is washed off the hair lock and neutralized. An apparatus for performing this method has a separate flat hair lock support (4) and a separate heating unit (5). The heating unit (5) has a hot plate (6) for heating the hair lock (2) placed on the protective foil (7). A timer device (17) for setting the heating time may be included in the apparatus.

### BACKGROUND OF THE INVENTION

1. Field of the Invention

The present invention relates to a method for changing color of hair locks and, more particularly, to a method for changing color of hair locks by means of a treatment composition under the influence of heat. It also relates to an apparatus for performing this method.

2. Description of Related Art

Methods and apparatus for changing color of hair locks by means of a treatment composition are known in various forms from well-known prior public use. For example, shorter hair treatment times for bleaching hair are produced by using a heat-radiating unit.

These methods have the following disadvantages.

The heat-radiating unit heats the entire head of the person who is treated. Because of that aspect of the known method only a comparatively small temperature increase of the treatment composition on the hair lock is possible, so that the treatment to change the color of the hair lock is comparatively very long (about 20 to 45 minutes).

The treatment times are comparatively long, even when the color of only a few hair locks is to be changed.

The energy consumption is high, since the heat-radiating unit must heat the entire head.

The hair lock being treated experiences an unpleasant pulling in the socalled plastic foil technique, in which a plastic foil cap is penetrated with a hook needle and the hair lock is pulled through it.

The treatment composition can reach undesirable locations.

### SUMMARY OF THE INVENTION

It is an object of the present invention to provide a method for changing color (providing colored accents) of hair locks by means of a treatment composition or treatment agent, which does not have the above-described disadvantages.

It is also an object of the present invention to provide an apparatus for performing the method according to the invention.

According to the invention the method for changing color of a hair lock by means of a treatment composition under the influence of heat includes:

a) placing a protective foil for the hair lock on a flat hair lock support;

b) placing the hair lock on the protective foil that is placed on the flat hair lock support;

c) after steps a) and b), applying the treatment composition to the hair lock;

d) after the applying of step c), folding a free portion of the protective foil and laying it on the hair lock treated with the treatment composition;

e) after step d), placing a hot plate on the hair lock and heating the hair lock with the hot plate for a predetermined time interval; and

f) subsequently rinsing the treatment composition from the hair lock and neutralizing, as needed.

In preferred embodiments of the method the treatment composition is a bleaching or oxidation hair dye composition.

An apparatus for performing the method of coloring hair locks or accenting portions of the hair includes

a flat hair lock support and an electrical heating unit which are separate from each other;

wherein the hair lock support comprises means for supporting a hair lock placed on or in a protective foil and the electrical heating unit comprises means for heating the hair lock placed on or in the protective foil and supported on the hair lock support for a predetermined heating time interval. The means for heating the hair lock includes a hot plate, preferably heated electrically with an electrical heating element, and means for bringing the hot plate into a position adjacent the hair lock placed on or in the protective foil on the hair lock support, so that the hair lock can be heated for the predetermined heating time interval.

In a preferred embodiment of the apparatus the hair lock support comprises a spatula including a comb-like device. The comb-like device comprises a first stationary row of first teeth, a second movable row of second teeth and an advantageously pivotable spring element for urging the second movable row away from the first stationary row of teeth. An operating element for moving the second movable row toward the first stationary row against the action of the spring element is also provided. The first teeth and second teeth are shaped so that the first teeth and the second teeth may be brought into coincidence or engagement with each other by applying a manual pressure to the operating element to move the spring element. Intervening spaces between the first and second teeth are thus opened so that hairs of the hair lock may be inserted or placed in the intervening spaces. Then the hair lock can be clamped in the hair lock support when the manual pressure on the operating element ceases.

A timer device can be provided in the electrical heating unit for setting the predetermined heating time interval. A signaling device is advantageously also provided to signal the expiration of the heating time interval optically and/or acoustically.

### BRIEF DESCRIPTION OF THE DRAWING

The objects, features and advantages of the invention will now be illustrated in more detail with the aid of the following description of the preferred embodiments, with reference to the accompanying figures in which:

FIG. 1 is a perspective view of an apparatus for performing a method of providing color accents to hair locks according to the invention, which comprises a flat hair lock support and a heating unit;

FIG. 2 is a cross-sectional view through the heating unit shown in FIG. 1;

FIG. 3 is a plan view of the heating unit shown in FIG. 2;

FIG. 4 is a side view of the heating unit in FIG. 2;

FIG. 5 is a bottom view of the heating unit in FIG. 2;

FIG. 6 is front view of the heating unit in FIG. 2;

FIG. 7 is a cutaway partial plan view of the hair lock support of FIG. 1;

FIG. 8 is a cross-sectional view taken through the hair lock support shown in FIG. 7 along the section line VIII-VIII, in the vicinity of a first row of teeth;

FIG. 9 is another cross-sectional view taken through the hair lock support along the section line IX-IX of FIG. 7;

FIG. 10 is a detailed cross-sectional view of a portion of the first row of teeth shown in FIG. 7;

FIG. 11 is a detailed side view of the first row of teeth shown in FIG. 9;

FIG. 12 is a plan view of a lower comb part with a second row of teeth, a spring element and an operating element;

FIG. 13 is a side view of the lower comb part shown in FIG. 12;

FIG. 14 is a plan view of the hair lock support according to FIG. 7, however with the lower comb part shown in FIGS. 12 and 13;

FIG. 15 is a cross-sectional view of the apparatus shown in FIG. 14 taken along the section line XV-XV in the vicinity of the first and second rows of teeth in an open position;

FIG. 16 is a plan view of the hair lock support similar to that of FIG. 14, but with the first and second rows of teeth in a closed position;

FIG. 17 is a cross-sectional view of the hair lock support similar to that of FIG. 15 and taken along the section line XVII-XVII of FIG. 16, but with the first and second rows of teeth in the closed position;

FIG. 18 is another cross-sectional view through the hair lock support shown in FIG. 16, but taken along the section line XVIII-XVIII;

FIG. 19 is a cross-sectional view showing the first and second rows of teeth according to FIG. 15, but with the hair locks or strands between the teeth of both rows with the rows of teeth in their open position;

FIG. 20 is a cross-sectional view showing the first and second rows of teeth according to FIG. 15, but with the hair locks or strands clamped between the teeth of both rows with the rows of teeth in their closed position;

FIG. 21 is a detailed partial cross-sectional view of teeth in the positions shown in FIGS. 19 and 20;

FIGS. 22 and 23 show two respective alternative embodiments for the shape of the teeth of the two rows of teeth and are similar to FIG. 21;

FIG. 24 is a plan view of the apparatus of FIG. 1 assembled from the hair lock support and heating unit;

FIG. 25 is a side view of the apparatus shown in FIG. 24; and

FIG. 26 is a detailed cross-sectional view of the apparatus shown in FIG. 24, which is taken along the section line XXVI-XXVI of FIG. 25 and in which the heating unit is in thermal contact with the hair lock wrapped or enclosed in a protective foil together with the treatment composition.

### DETAILED DESCRIPTION OF THE INVENTION

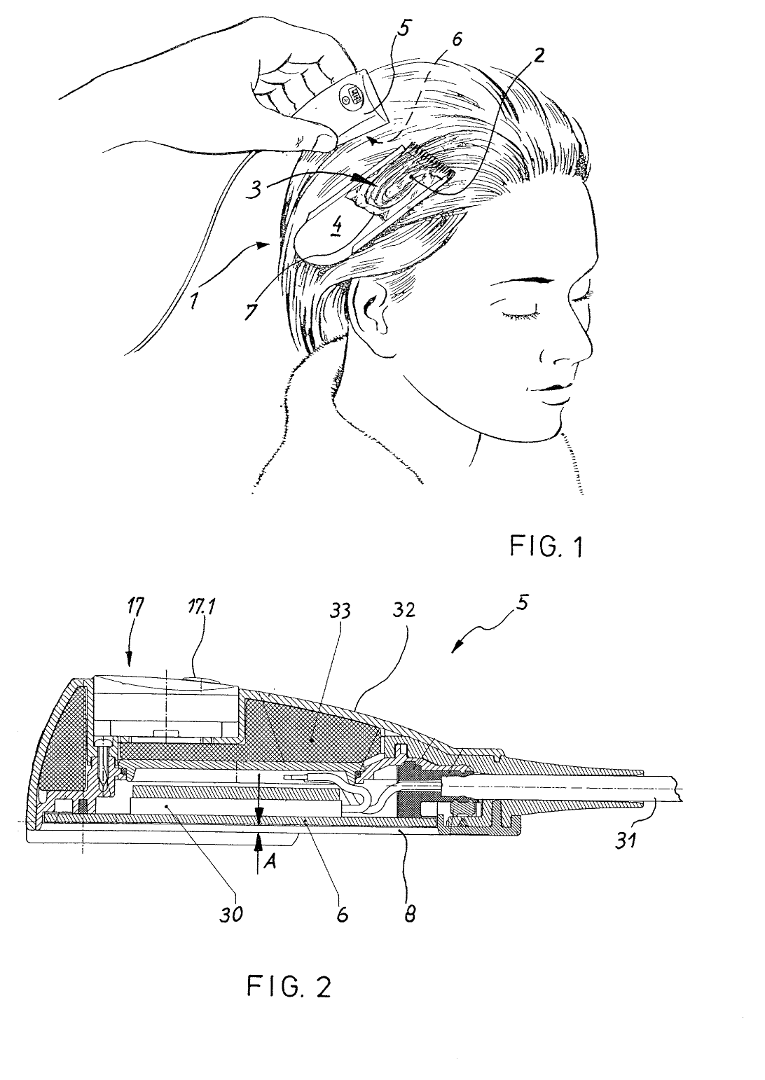

An apparatus 1 for performing the change of color of the hair lock is described in detail here first. FIG. 1 shows an apparatus 1 for changing color of hair locks by means of a treatment agent or composition 3. The apparatus comprises a planar or flat hair lock support 4 and an electrically heated heating unit 5, which are separate from each other. The heating unit 5 has a flat hot plate 6, which is heated in operation. The hair lock 2 is enclosed in a plastic foil 7 and placed or laid on the hair lock support 4. The hot plate 6 with the heating unit 5 is then put in thermal contact with or adjacent to the hair lock 2 and the hair lock 2 is heated with it for a predetermined heating time interval during the heat treatment. A heat resistant and transparent (plastic) protective foil 7 preferably encloses the hair lock 2 during the heat treatment.

FIG. 2 shows the heating unit 5 in cross-section. A spacer member 8 is provided for keeping the hot plate 6 a predetermined distance from the flat side of the hair lock support 4 during the treatment, so that the treatment composition 3 cannot be pressed from the plastic foil 7 by the hot plate 6. The spacing A amounts to about 2 mm. An electrical heating element 30 is provided for heating the hot plate 6. The electrical heating element 30 is supplied with electrical energy by a cable 31. The heating unit 5 is provided with a heating unit housing 32, which is protected from heat from the heating element 30 by means of a heat insulating body 33. The heating unit 5 also includes a timer device 17. The duration of the heating or heat treatment is set or controlled by means of the timer device 17. The timer device 17 may be provided with a signaling device 18, which can be activated by a timer device switch 17.1, which can be an on/off button. The signaling device 18, when activated, produces an optional optic and/or acoustic signal when the heating time interval expires.

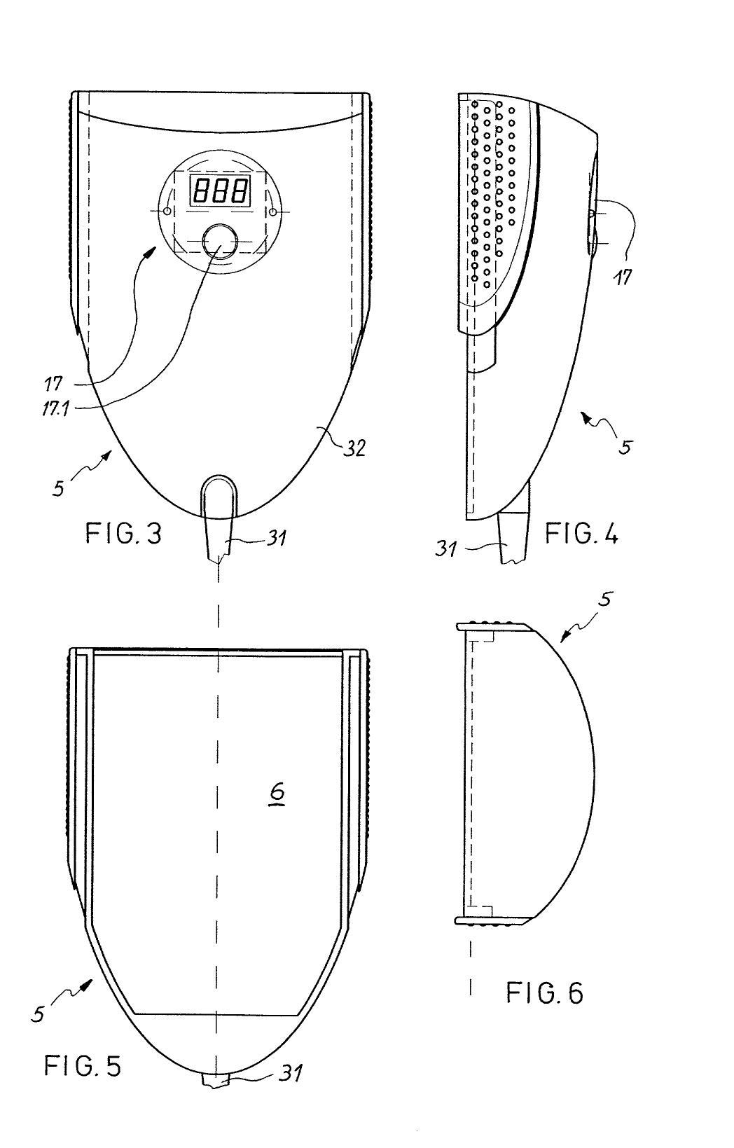

FIGS. 3 to 6 show different views of the heating unit.

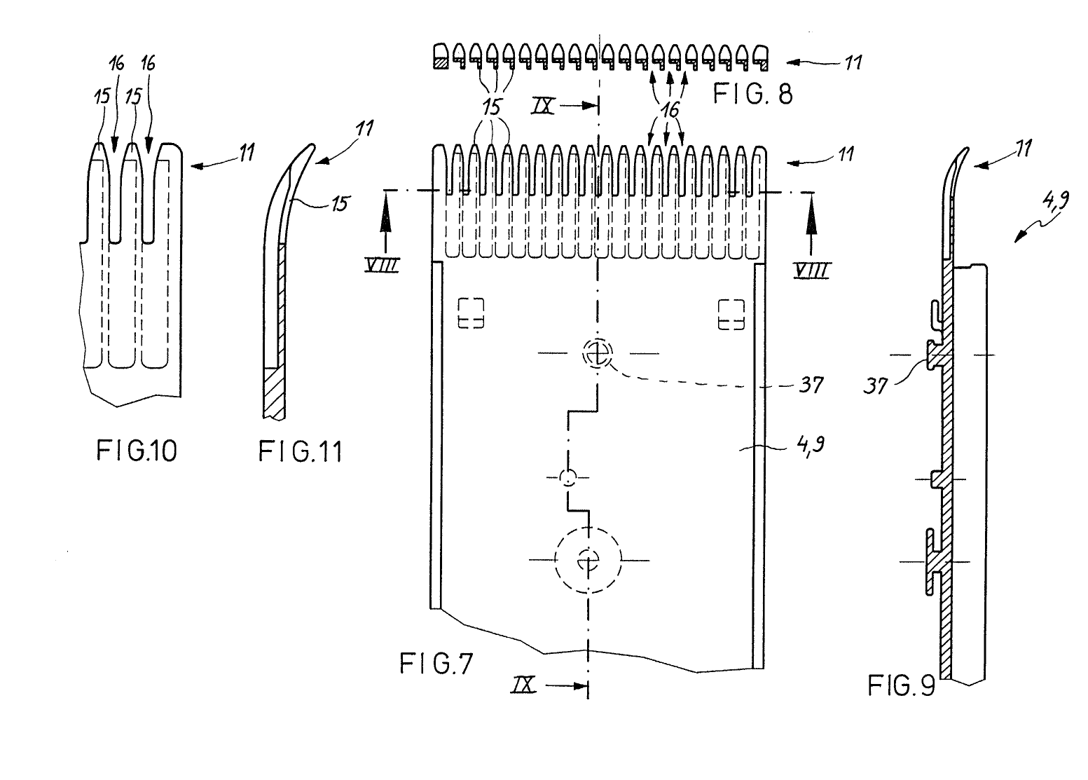

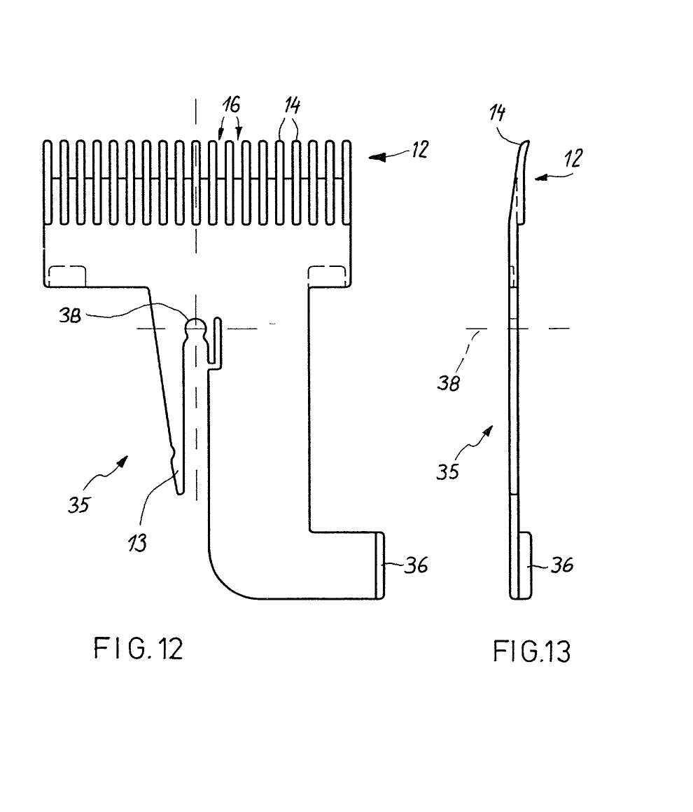

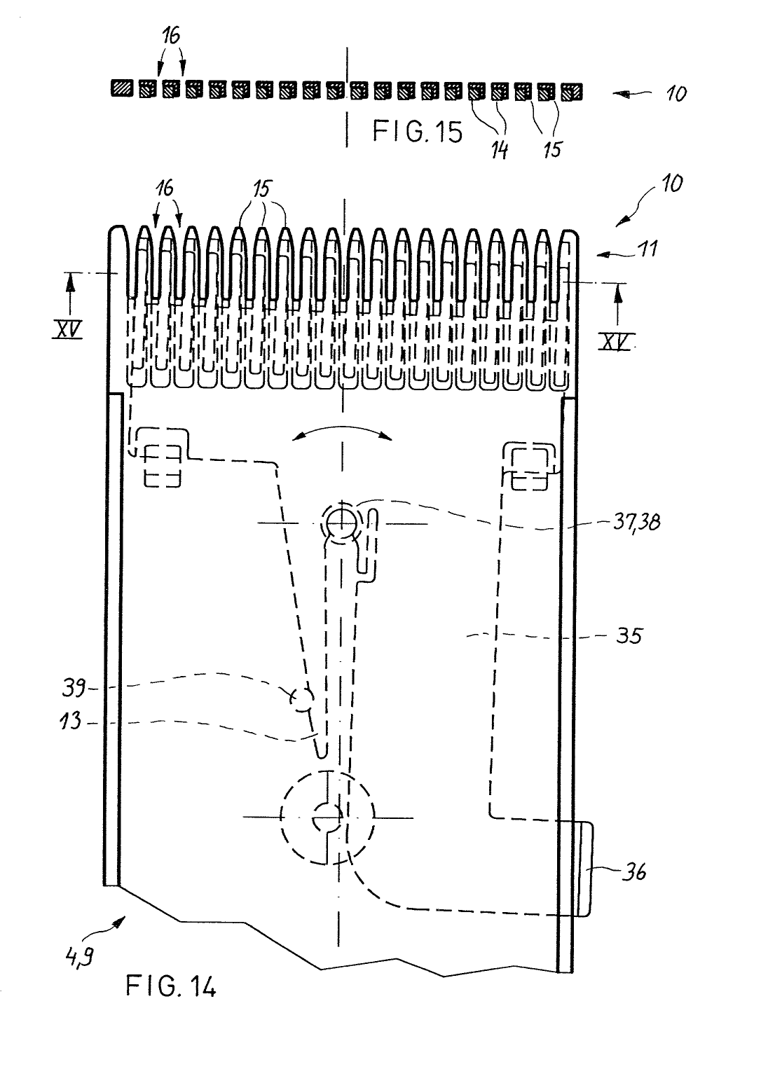

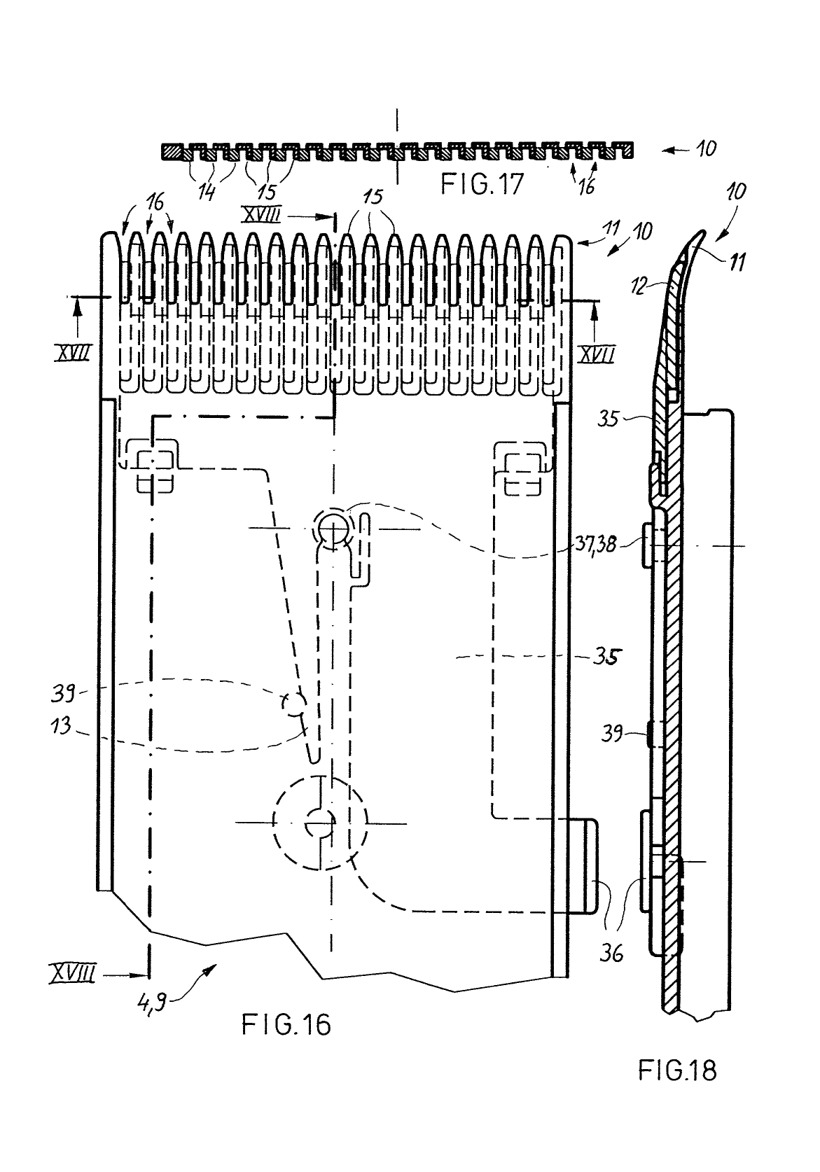

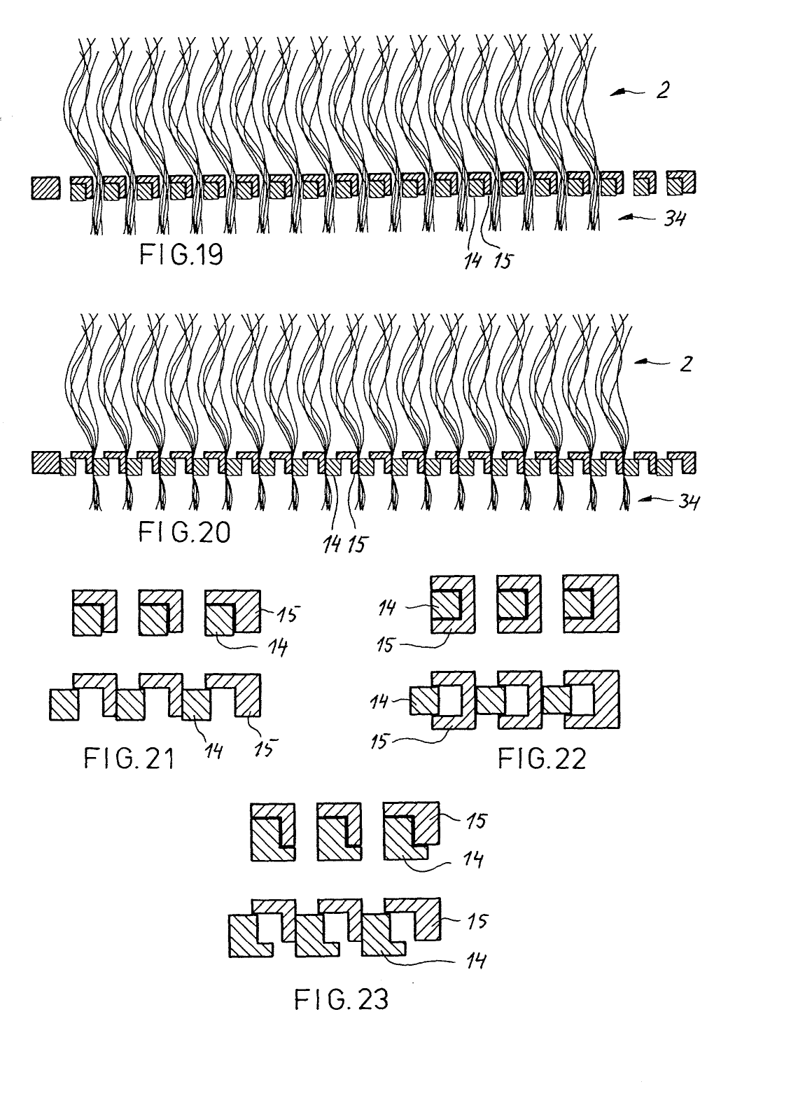

The hair lock support 4 is provided with a stationary comb 10 for better handling (FIGS. 14 to 23) on one end or the other, which is formed like a spatula 9 (FIG. 7). The spatula 9 can be clamped in the hair roots 34 (FIGS. 19 and 20) or in the hair locks in a detachable or releasable manner. For this purpose the spatula 9 has a first stationary row 11 of first teeth 15 (FIG. 7 to 11), which correspond in their shape to second teeth 14 of a spring-loaded second row 12 of teeth (FIGS. 12 and 13). The second row 12 of second teeth 14 are provided on a lower comb part 35 arranged under the first row 11. A spring element 13 urges the flanks or sides of the second teeth 14 of the second row 12 against the flanks or sides of the first teeth 15 of the first row 11 of teeth (FIG. 16). The respective teeth of both rows 11,12 are brought into coincidence or engagement with each other by means of pivot bearing 37,38 (FIG. 14) when an operating element 36 of the lower comb part 35 is manually pressed. When the respective rows 11,12 of teeth coincide or engage the intervening spaces 16 between the teeth of the first and second rows are open so that hairs of the hair lock 2 can be inserted or placed in the intervening spaces 16. The pivot bearing 36,37 is a snap coupling, so that the lower comb part 35 may be easily removed for cleaning both parts (stationary comb 10 and lower comb part 35). The spring element 13 bears on counter member 39, which is arranged on the underside of the stationary comb 10. The spring element 13 is formed from plastic material in one piece with the lower comb part 35. In alternative embodiments a separate spring element made of metal may be used.

In order to thread hairs of the hair lock 2 into the stationary comb 10, the second teeth 14 of the lower comb part 35 must be pushed under the first teeth 15 of the upper first row 11. The stationary comb 10 is open now and the hairs can be inserted (FIG. 19). When the hairs are in the stationary comb 10, the operating element 36 is released. The spring element 13 then presses the teeth 14 of the second row 12 into the intervening spaces 16 between the first row 15 of teeth. The hairs now are clamped between the teeth 14 of the lower comb part 35 and the teeth 15 of the upper first row 11 (FIG. 20). The hair lock 2 is now fixed or rigidly connected with the spatula 9. The release of the spatula 9 from the hair lock 2 occurs by operation of the operating element or button 36 and removal of the hair lock 2.

So that the hair always contacts the correct sides of the teeth 14,15, the first teeth 15 of at least the upper first row 11 are L-shaped (FIG. 21). The second teeth 14 of the second row 12 engage with the teeth 15 of the first row 11. The first teeth 15 may also be almost exactly U-shape, however it is more difficult to make the first teeth 15 in this shape (FIG. 22).

An additional advantage results from the sealing action of the stationary comb 10. When the stationary comb 10 is attached to a hair lock 2, the intervening spaces 16 are closed. Thus chemical preparations cannot reach the scalp when they are subsequently applied. This sealing action can be further increased when at least one of the contacting teeth flanks is coated with an elastic material. This coating would also increase the friction between the teeth 14,15 and the hair so that the adherence to the hair lock 2 is increased.

FIG. 21 is an enlarged view of teeth 14,15 of the first and second rows 11,12 in a first and second position in the above-described embodiment. FIGS. 22 and 23 shows two alternative embodiments in which the teeth 14,15 of the first and second rows have different shapes.

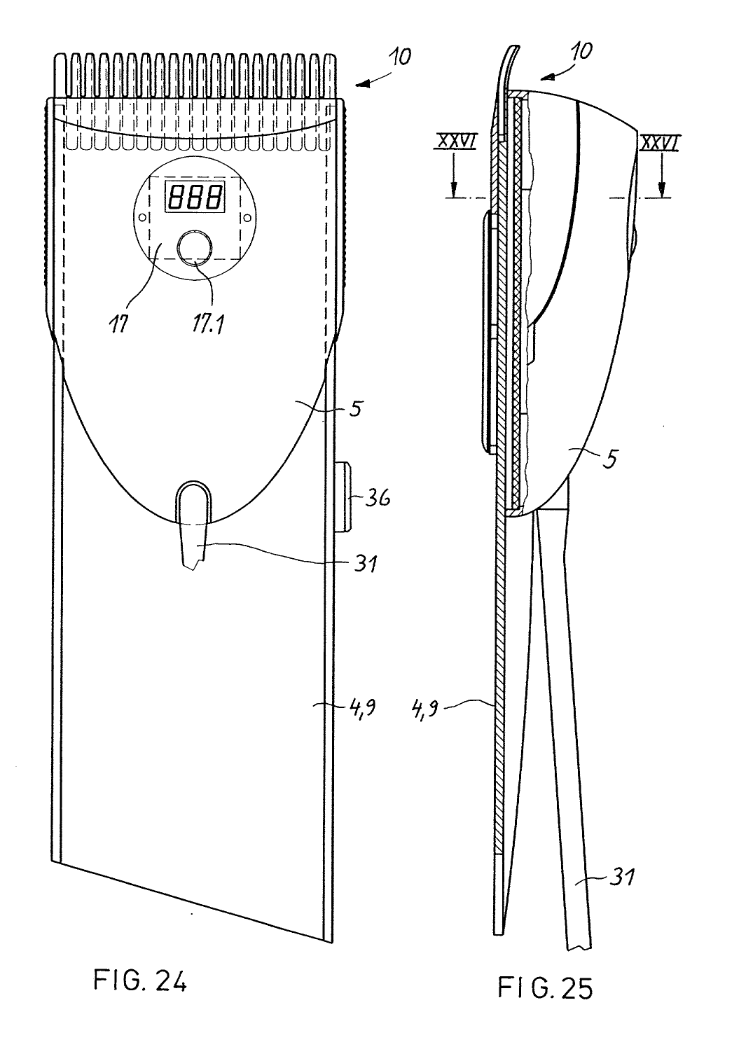

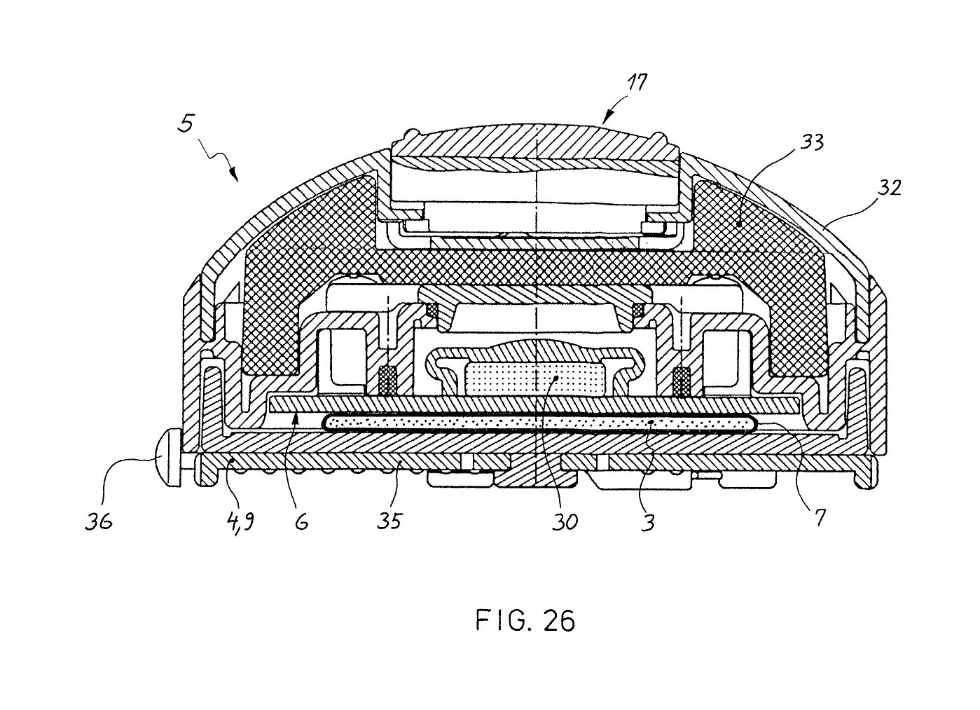

The apparatus 1 is shown in FIG. 24 with the heating unit 5 on the hair lock support 4 (spatula 9) in a position to perform the heat treatment. A corresponding side view is shown in FIG. 25. FIG. 26 shows a cross-sectional view taken through the apparatus 1 taken along the section line XXVI-XXVI of FIG. 25.

The method of the invention changes the color of hair strands of a hair lock 2 or provides color accents to hair locks 2 of human hair. The method starts by placing or laying a protective foil 7 on a flat hair lock support 4 and subsequently placing or laying the hair lock 2 on the protective foil 7. Then a treatment agent or preparation 3 is applied to the hair lock 2 and after that a free part of the protective foil 7 is folded over the hair lock, so that it contacts it. After that a hot plate 6 is put on the hair lock 2 for a predetermined heating time interval. Subsequently the hair treatment composition 3 is rinsed from the hair lock 2 and is neutralized if necessary.

The procedure for performing the method for changing color of hair locks 2 by means of a treatment agent or composition 3 together with applied heat takes place using the apparatus 1. The method according to the invention comprises:

a) placing or laying a protective foil on the hair lock support 4;

b) dividing the hair lock 2;

c) placing or clamping the hair lock 2 in or on the hair lock support 4 and laying or placing the hair lock 2 on the protective foil 7;

d) applying the treatment composition (bleaching/dyeing composition) 3 on the hair lock 2, for example by means of a brush;

e) folding the protective foil 7 and putting the hot plate 6 at a predetermined position on the hair lock support 4 over the hair lock 2 and starting the timer device 11 (whereby the hair lock 2 is heated to about 85 degrees);

f) according to the desired degree of treatment (degree of lightening or coloring) the heating unit 5 is removed after about 1 to 2 minutes from the hair lock 2; and

g) the hair lock 2 is washed and neutralized, after the upper side of the protective foil 7 is unfolded or removed entirely. The washing out (rinsing) of the hair lock 2 takes place, preferably on the spatula 9, which then acts as a channeling device. After that the spatula 9 is separated from the hair roots 34 and can be used for additional hair locks 2 to be treated.

Between 2 and 5 minutes are required for these process steps. In the same way subsequently special color effects or accents can be obtained on the same hair lock with direct-dyeing dyes. Also oxidation hair dyed colors may be produced in the above-described manner.

When it is stated that the heating unit 5 and hair lock support 4 are separate from each other in the appended claims it means that they are not physically connected or attached to each other. However the hair lock support 4 is formed so that the heating unit 5 can be placed on it or engaged with it over the hair lock enclosed by the protective foil as described above for the purpose of performing the method of the invention.

The disclosure in German Patent Application 100 62 132.5 of Dec. 13, 2000 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in a method and apparatus for changing color of hair locks, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A method for changing a color of a hair lock (2) by means of a treatment composition (3) under the influence of heat, said method comprising the steps of:
a) first placing a protective foil (7) for the hair lock on a flat hair lock support (4);
b) placing the hair lock (2) on the protective foil (7) placed on the flat hair lock support (4);
c) after the placing of step a) and the placing of step b), applying the treatment composition (3) to the hair lock (2);
d) after the applying of step c), folding a free portion of the protective foil (7) and laying said free portion on the hair lock (2) treated with the treatment composition (3);
e) after the folding and laying of step d), placing a hot plate (6) on the hair lock (2) and heating the hair lock (2) with the hot plate (6) for a predetermined time interval; and
f) subsequently rinsing the treatment composition (3) from the hair lock (2) and neutralizing, as needed.

2. The method as defined in claim 1, wherein the flat hair lock support (4) has means (11,12,13) for clamping the hair lock (2) and further comprising clamping said hair lock (2) in said hair lock support (4) with said means for clamping.

3. The method as defined in claim 1, wherein said treatment composition (3) is a bleaching composition, an oxidation dye composition or a direct-dyeing hair dye composition and the predetermined time interval is about 1 to 2 minutes.

4. The method as defined in claim 1, wherein said hair lock is heated at about 85 degrees.

5. An apparatus for changing a color of a hair lock by means of a treatment composition (3) under influence of heat, said apparatus (1) comprising
a flat hair lock support (4) and an electrical heating unit (5), wherein said heating unit (5) and said hair lock support (4) are separate from each other;
wherein the hair lock support (4) comprises means for supporting a hair lock placed on or in a protective foil (7) and the electrical heating unit (5) comprises means for heating the hair lock (2) placed on or in the protective foil (7) and supported on the hair lock support (4) for a predetermined heating time interval; and
wherein the means for heating the hair lock includes a hot plate (6) and means for bringing the hot plate (6) to a position adjacent the hair lock (2) placed on or in the protective foil (7) on the hair lock support (4), so that the hair lock is heated when the electrical heating unit (5) is turned on.

6. The apparatus as defined in claim 5, further comprising the protective foil (7) and wherein the protective foil (7) comprises a heat-resistant and transparent plastic material.

7. The apparatus as defined in claim 5, wherein the hot plate (6) is provided with a spacer member (8) for holding the hot plate (6) at a predetermined distance (A) from an opposing side of the hair lock support (4).

8. The apparatus as defined in claim 5, wherein the hair lock support (4) comprises a spatula (9).

9. The apparatus as defined in claim 8, wherein said spatula (9) comprises a stationary comb (10) at one end thereof and said stationary comb (10) includes a first stationary row (11) of first teeth (15).

10. The apparatus as defined in claim 8, wherein said spatula (9) has a first stationary row (11) of first teeth (15), a second movable row (12) of second teeth (14), a spring element (13) for urging said second movable row (12) of said second teeth away from said first stationary row (11) and an operating element (36) for moving said second movable row (12) of said second teeth toward said first stationary row (11) of said first teeth (15) against the action of said spring element (13) and wherein said first teeth (15) and said second teeth (14) are shaped so that said first teeth and said second teeth are brought into coincidence or engagement with each other by applying a manual pressure to said operating element (36), whereby intervening spaces (16) between said first teeth and said second teeth are opened so that hairs of said hair lock (2) may be inserted or placed in said intervening spaces (16) and clamped in the hair lock support (4) when said manual pressure on said operating element (36) ceases.

11. The apparatus as defined in claim 5, wherein said means for heating the hair lock (2) includes a timer device (17) for setting said predetermined heating time interval.

12. The apparatus as defined in claim 11, wherein said timer device (17) includes a signaling device (18) for signaling when said heating time interval expires.
